(19) United States
(12) Patent Application Publication
(10) Pub. No.: US 2022/0384036 A1
(43) Pub. Date: Dec. 1, 2022

(54) SCALABLE ARCHITECTURE SYSTEM FOR CLINICIAN DEFINED ANALYTICS

(71) Applicant: Vital Connect, Inc., San Jose, CA (US)

(72) Inventors: Ian Felix, San Jose, CA (US); Gabriel Nallathambi, San Jose, CA (US); Nersi Nazari, San Jose, CA (US)

(21) Appl. No.: 17/335,911

(22) Filed: Jun. 1, 2021

(51) Int. Cl. G16H 50/20; G16H 40/67; G16H 10/60; G16H 80/00

(52) U.S. Cl. CPC G16H 50/20; G16H 40/67; G16H 10/60; G16H 80/00

(57) ABSTRACT

A method and system of a scalable modular architecture for enabling clinicians to define clinical inputs, operators, and notifications on a per patient and enterprise basis for screening any pathological condition per the clinical practice

### BACKGROUND

Clinicians utilize decision support systems to assist in early detection of different disease types and provide timely intervention. Clinical decision support systems need to provide transparent, understandable and explainable decisions. Unlike opaque decisions resulting from black-box algorithms, transparent algorithms enhance the trust of clinicians on the automated decision support platforms. This requires that clinicians must know and understand why a support system decision was triggered. Furthermore, seamless integration of measurement and lab data is needed for timely decision making systems that can be used in a standard hospital or home setting to monitor patients for improvement or decline of their diseases.

The present application overcomes the limitation of traditional decision making systems used in a standard hospital or home setting to monitor patients for improvement or decline of their disease. The present disclosure provides a scalable modular architecture that can be integrated into clinical workflow and enables clinicians to explicitly define analytics for triggering reliable, transparent and explainable decision support systems.

### SUMMARY

In one example embodiment, a method for a scalable modular architecture for clinician defined analytics is described, including: measuring, by one or more sensors of one or more devices, one or more clinical measurements of a patient; aggregating, by a command center, the one or more clinical measurements; extracting, by the command center, clinical measurement vectors from the aggregated clinical measurements for one or more decision system; selecting, by the command center, appropriate clinical measurements relevant to triggering one or more decision systems from a pool of incoming data; and performing computations, by the command center, to define an analytics engine per analytics specifications and operations.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

In the brief description that follows, embodiments are described as illustrations only since various changes and modifications will become apparent to those skilled in the art from the following detailed description. The use of the same reference numbers in different figures indicates similar or identical items.

FIG. 1 illustrates an example block diagram for implementing one or more embodiments of the system and method for scalable clinician defined analytics.

FIG. 2 illustrates an example block diagram of the architecture for clinician defined analytics.

FIG. 3 illustrates an example block diagram of the Clinician portal and defined analytics engine.

FIG. 4 illustrates an example block diagram of the vector layer.

FIG. 5 illustrates an exemplary Boolean process.

FIG. 6 illustrates an exemplary scoring process.

FIG. 7 illustrates an exemplary dynamic process.

FIG. 8 illustrates an exemplary mixed process.

FIG. 9 illustrates an example block diagram of the assist engine.

FIG. 10 illustrates an example block diagram of a computing device.

### DETAILED DESCRIPTION

In the following detailed description, reference is made to the accompanying drawings, which form a part of the description. In the drawings, similar symbols typically identify similar components, unless context dictates otherwise. Furthermore, unless otherwise noted, the description of each successive drawing may reference features from one or more of the previous drawings to provide clearer context and a more substantive explanation of the current example embodiment. Still, the example embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein and illustrated in the drawings, may be arranged, substituted, combined, separated, and designed in a wide variety of different configurations, all of which are explicitly contemplated herein.

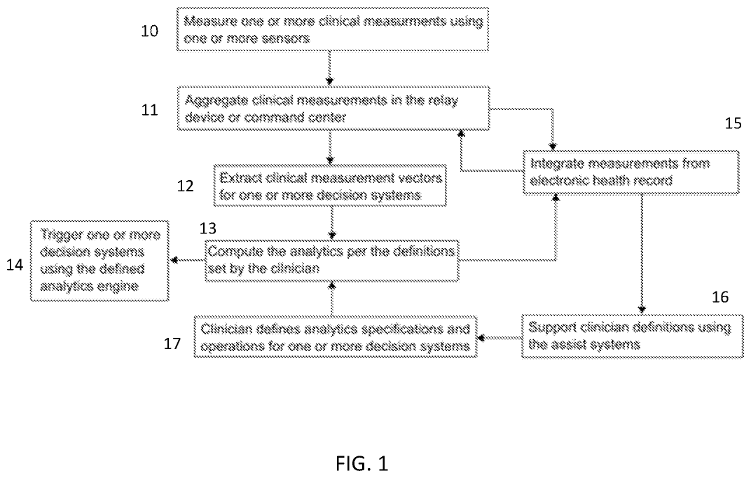

FIG. 1 shows an example illustration for implementing one or more embodiments of the method and scalable modular architecture for clinician defined analytics. As illustrated in FIG. 1, for example, one or more sensors of one or more devices measure one or more clinical measurements at 10. The one or more clinical and lab measurements are aggregated and/or accumulated in a relay device or command center at 11. Clinical measurement vectors are extracted from the aggregated clinical measurements for one or more decision systems at 12. That is at 12, appropriate clinical measurements relevant to triggering one or more decision systems is selected from the pool of incoming data. Computations are performed to compute and define the analytics engine per the analytics specifications and operations defined and set by the clinician at 13. One or more decision support systems are triggered using the defined analytics engine at 14.

The clinician defined analytics system also includes the option of assisting clinicians in selecting the definitions of the analytics specifications and operations by learning optimal decisions based on the outcomes of prior definitions and operations. AIII measurements from the relay process of 11 may be integrated into the Electronic health record (EHR) at 15. The clinician defined analytics system supports clinician definitions using the assist systems at 16. Clinicians define analytics specifications and operations for one or more decisions systems at 17. The clinicians definitions of 16 are input for the analytics computations of 13. The analytics computations of 13 are also inputted to and integrated into the EHR of 15.

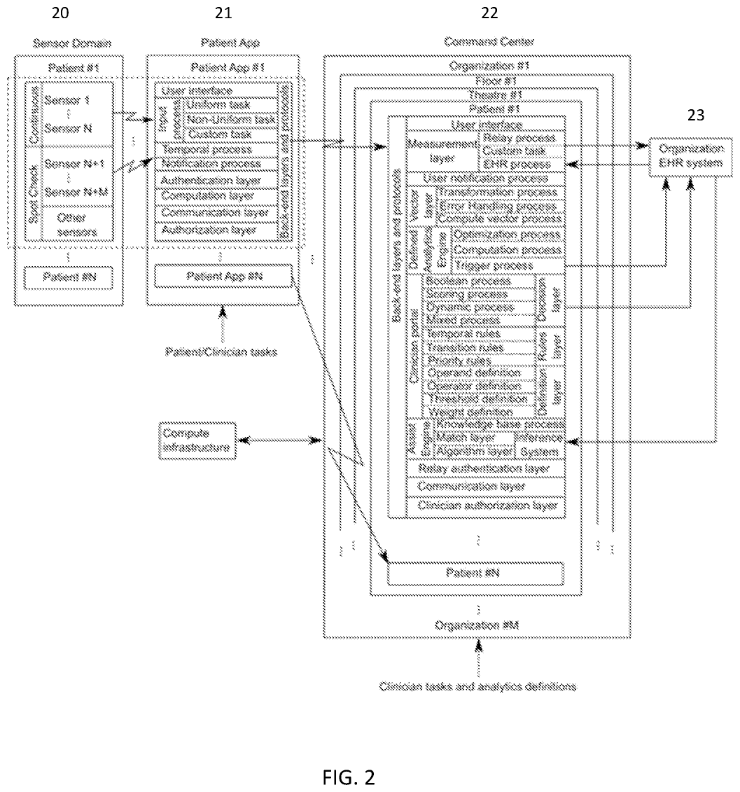

Additionally, the system is capable of interpreting a “digital prescription” from the attending clinician to accomplish:
1. To precompute various averages and metrics of vital signs per clinician instruction so it can respond faster.
2. Per complexity of the prescription, explicitly or implicitly, to communicate with the underlying computing structure (usually “cloud computing”) to bring the necessary resources to respond per clinician instructions.
3. The clinician may further instruct the system to make above adjustments based in part of whole on patient's electronic health record.
4. The system will add in modular fashion per digital prescription computing hardware and software for a given patient or population of patients. The system is also capable of releasing those resources once the patient(s) is(are) discharged.
   More details of the above architecture for clinician defined analytics system is described by a block diagram representation in FIG. 2.

FIG. 2 illustrates the architecture for clinician defined analytics system includes four main components as follows: sensor domain 20, patient app 21, command center 22, and electronic health record (EHR) 23. Each component is organized into one or more modules that ensures scalability and ease of integration. Further, each module is categorized into the following levels of hierarchy as follows: engine, system, layer, process and tasks. Each level of hierarchy may be a self-contained functionality in a microservices architecture with processing done in one or more distributed compute nodes with multiple redundancies. Each component of the architecture is described as follows.

As shown in FIG. 2, the sensor domain 20 system may include one or more sensors for measuring clinically relevant variables of one or more patients. The system may be self-contained or separate devices that are placed on one or more locations of the body, or implanted inside the body. The sensor systems which are in contact with the body or non-contact are within the scope of this invention.

The sensor devices of sensor domain 20 are capable of taking continuous or spot check automated measurements, and manual spot check measurements. The continuous measurements are performed by 1 . . . N number of sensors in devices such as, for example, vital sign monitors, pacemakers, spinal cord stimulators, insulin pumps, and blood glucose meters which include and are within the scope of this invention. The manual spot check measurements are performed by N+1 . . . N+M number of sensors in devices such as, for example, thermometers, temperature probes, spirometers, blood pressure monitors, weighing scale, pulse oximetry are within the scope of this invention. The sensor domain 20 may also monitor #1 . . . #N Patients.

The devices of Sensor Domain 20 may communicate with Patient App 21 (which is deployed in, for example, a relay or mobile device such as phone or tablet) via communication technologies such as, for example, WIFI, Bluetooth, ZigBee, etc. to ensure encrypted, secure communication. Sensor Domain 20 may include transceivers and receivers for communication with Patient App 21. Each communication initiates a unique patient session after authentication of the user.

As shown in FIG. 2, the patient app 21 includes an input process that includes tasks for distinguishing and processing one or more sensing modalities from sensor domain 20 during the decryption processes based on uniform or non-uniform frequency of measurement. Patient/Clinician tasks may be entered via the Patient App 21. Additionally, notification processes with pre-defined frequencies are used to remind users to take measurements, and custom tasks of input process determine the processing modality (uniform task or non-uniform task) of the manual measurements. Temporal process ensures each incoming sample of data is associated with the unique timestamp of required resolution, and enables grouping of information in a time window.

Authorization layer of Patient App 21 has secure and password protected patient portal, technician portal and nurse portal with appropriate controls for entering clinical measurements. Computation layer of Patient App 21 allows processing at the edge extract semantic information and remove redundancies in the data. Communication layer of Patient App 21 has standard processes that decrypts the information from the sensor domain and displays them in the user interface of the app. The communication layer further compresses and encrypts information for transmitting the secured data to the command center using communication technologies such as WIFI, cellular, etc. The authentication layer uses protocols to communicate with the sensor domain and the command center to ensure each patient session is unique.

As shown in FIG. 2, the command center 22 is organized in layers of hierarchy such as organization, floors, theatres, and patients. The measurement layer of the command center has relay processes to receives information from the patient app 21 (relay device), EHR process to receive input such as lab results and other measurements from an Electronic Health Record (EHR). Additionally, all measurements from the relay process have the option of being integrated into the EHR. Clinician authorization layer has secure and password protected clinician portal, technician portal and nurse portal with appropriate controls for entering clinical measurements (organized by the custom task of measurement layer), and user notification processes with pre-defined frequencies to remind users to enter the measurements.

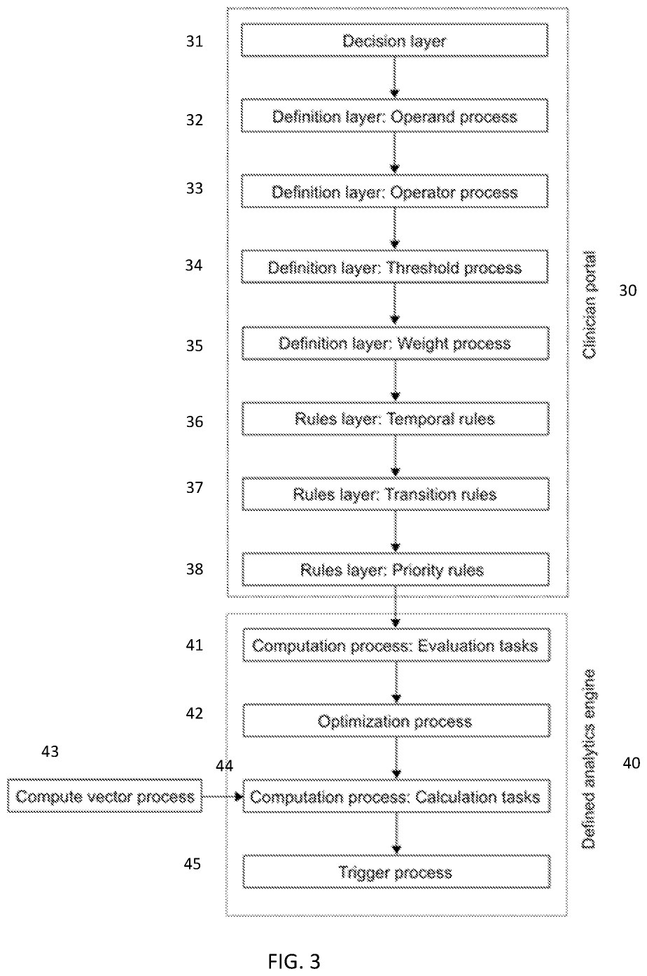

As shown in FIG. 3, a clinician portal 30 enables setting of the definitions by the clinician for the specifications and operations of the analytics. Further, FIG. 3 shows the sequence of steps involved in setting the decisions, rules, and definitions by the clinicians for triggering the decision support system. The first step is setting, by the clinician portal 30, the processes of the decision layer 31 for defining the explicit explanatory mathematical structure of the decision support system. Then, the operands required for the decision support system is defined in the definition layer (definition layer: operand process 32). The operand may be sensor inputs such as vital sign measurements, lab results such as blood parameter measurements, and other measurements. Subsequently, the operator used in the decision support system are defined in the definition layer where the operators may be arithmetic operators such as addition, subtraction, multiplication or division, logical operators such as AND, OR, negation, etc., relational operators such as greater than, less than, or equal to, etc., statistical operators such as mean, median, mode, variance, standard deviation, etc., or functional operators (definition layer: operator process 33). Threshold process in the definition layer provides the basis for comparison for interpreting the clinical measurements of a particular patient and may be an absolute value, a range or an interval that is deemed normal for a given clinical measurement in healthy persons or abnormal in pathological conditions (definition layer: threshold process 34). Weight process in the definition layer is used to assign various weights according to the levels of importance for the processing elements (definition layer: weight process 35). Temporal rules in the rules layer define the instances and windows of processing, and may be exact time i.e., every sample t_i, multiple points in time, overlapping or non-overlapping time windows (rules layer: temporal rules 36). Transition rules in the rules layer specify the definition of transitions to next state based on the current state and other inputs (rules layer: transition rules 37). Priority rules in the rules layer define the order of precedence for computations using parenthesis (rules layer: priority rules 38).

As further shown in FIG. 3, the defined analytics engine 40 is used to evaluate, optimize, compute, and trigger the decision support system based on the definitions of the clinicians. The evaluation tasks include evaluation and compilation of the definitions into a mathematical form that can be used for computation (computation process: evaluation tasks 41). The verification of the syntax may involve automated compilation rules or a manual input table of operand values that can be run through the mathematical form to ensure it gives the right output. The optimization process 42 includes minimization of the states or declarative statements by reducing the unreachable states or statements. The computation process uses the data vectors for the operands (compute vector process 43) and the optimized mathematical structure for computing the elements of the decision support system (computation process: calculation tasks 44). Trigger process 45 are used to trigger alerts based on the output of one or more decision systems.

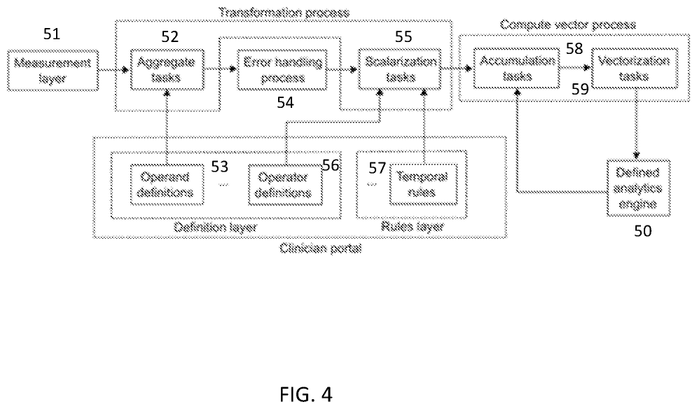

As illustrated in FIG. 4, a vector layer is described. The vector layer operates on the uniform and non-uniform inputs from the measurement layer 51 to form the vector of operand values that will be used for computation by the defined analytics engine 50. Aggregate tasks 52 involve extracting the measurement values of the operands defined by the clinician (operand definitions 53). Error handling process 54 involve tasks for processing and handling of outliers, missing entries, and incorrect entries. Scalarization tasks 55 involve computing a scalar value from a group of sample values of a given clinical measurement. Further, scalarization tasks 44 may use statistical operators (operator definitions 56) to transform the group of values into a scalar in the case of uniform inputs, or nearest available sample value in the case of non-uniform inputs. Further still, scalarization tasks 55 may be driven by the time tasks of the temporal process (temporal rules 57) i.e., whenever available, or time-windows based on prior time window or outcome. The scalar value of each clinical measurement is accumulated (accumulation tasks 58) to form the vector of operands (vectorization tasks 59) that will be used by the defined analytics engine 40.

The invention further includes the following embodiments of the decision layer 31.

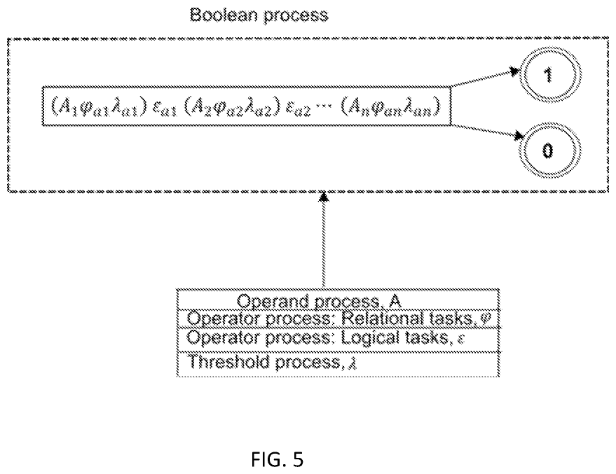

FIG. 5 shows an exemplary embodiment of the decision layer 31. For example, FIG. 5 discloses a Boolean process that defines a mathematical decision structure whose result (1 or 0) may trigger a single alert. The process begins by comparing operands A to threshold A using relational operators cp. The results of these individual operations can be combined using logical operators E to determine if the alert should be triggered (in case of 1) or not (in case of 0).

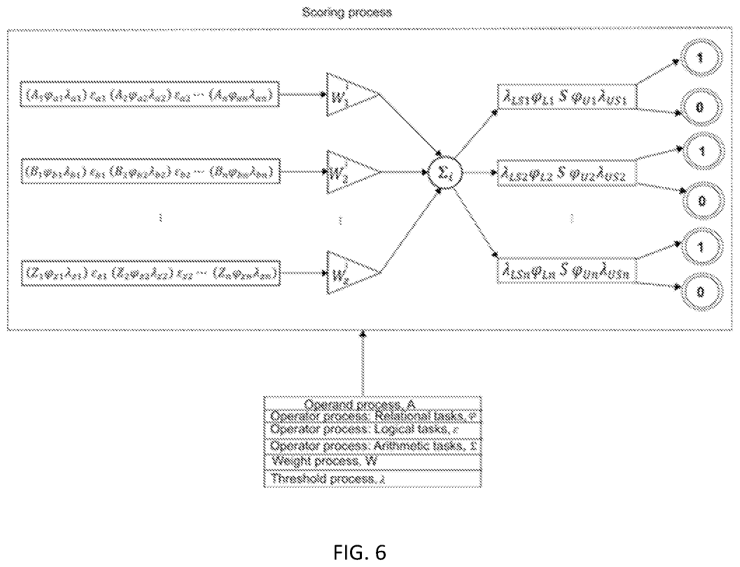

FIG. 6 shows another exemplary embodiment of the decision layer 31. For example, FIG. 6 discloses a scoring process that defines a mathematical decision structure whose result generates scores that may trigger a multiple alert. The process begins by computing multiple statements where each statement is a Boolean process i.e., it outputs 1 or 0 by comparing operands A to threshold λ using relational operators φ and combining operations using logical operators ε. Each statement is assigned different clinical levels of importance by multiplying weight W. The weighted results are then combined using arithmetic operators Σ to generate score S. The score is then compared to multiple lower and upper thresholds using relational operators to generate one or more alerts

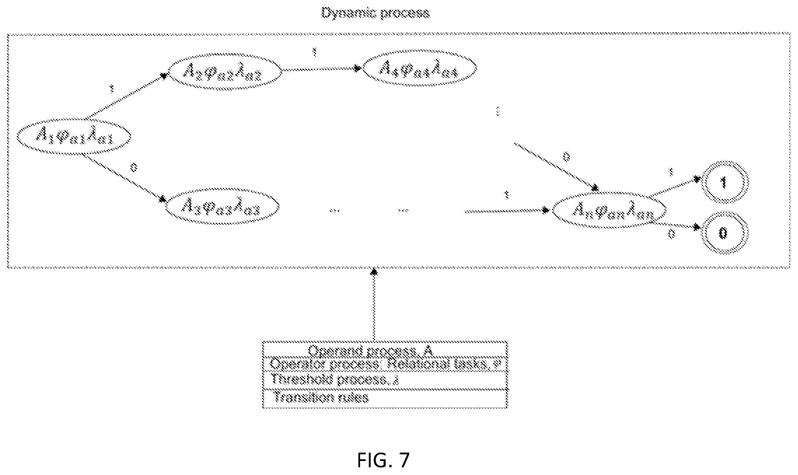

FIG. 7 shows another exemplary embodiment of the decision layer 31. For example, FIG. 7 discloses a dynamic process that defines a mathematical decision structure where the process transitions from one state to next state until it reaches the final state that may trigger a single alert. The process begins by evaluating operands A compared to threshold λ using relational operators φ to generate output 1 or 0. Based on the output, the process transitions from one state to next state using the transition rules defined by the clinician till the final state is reached and alert is triggered.

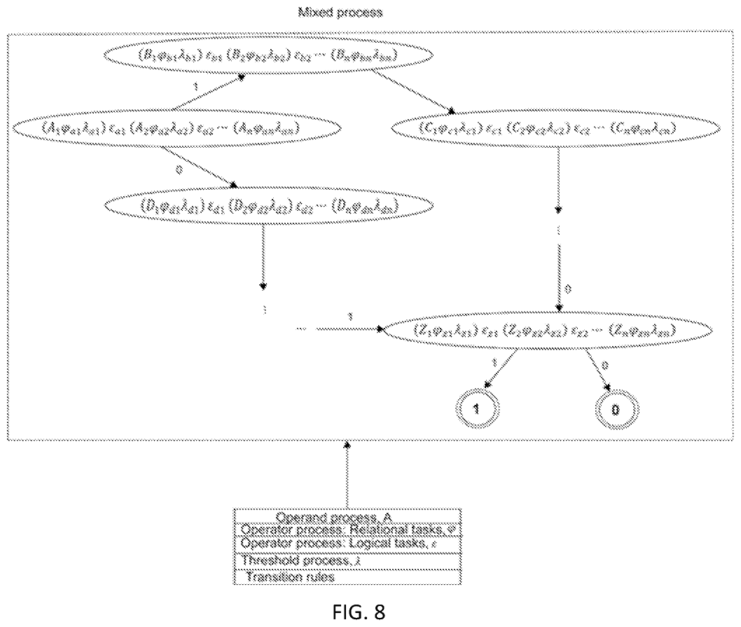

Another exemplary embodiment of the decision layer 31 includes a mixed process that defines a heterogeneous mathematical decision structure that is a combination of the above processes. For example, FIG. 8 shows a mixed process which is a dynamic process with each state being a Boolean process.

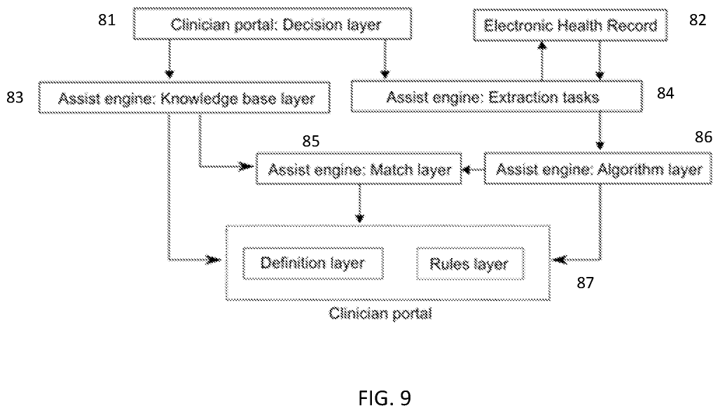

FIG. 9 shows an Assist Engine that supports clinicians to select optimal definitions and rules for a particular condition of a patient. The assist tasks may be condition-specific or patient-specific or both. An exemplary embodiment of condition-specific tasks may query a database containing the knowledge base 83 to provide the clinically acceptable definitions and rules 87 for the particular condition and decision layer 81. An exemplary embodiment of patient-specific tasks may involve extraction tasks 84 to obtain prior definitions and operations set by the clinician for the patient from the EHR 82 and algorithm process 84 to learn optimal decisions/rules based on the prior outcomes for the particular patient. An exemplary embodiment of the combination of condition-specific tasks and patient-specific tasks may be matching 84 the condition to the knowledge base 83 and the patient to learned optimal decisions/rules based on the prior outcomes, and provide comprehensive information to the clinician to set the definition and rules in the clinician portal. The output of the Assist Engine is intended for support purposes only and, at any point, the architecture provides the clinician with the ability to over-ride the assist engine's support for definitions and rules 87.

Thus, the disclosed modular architecture enables clinicians to define clinical inputs, operators, and alarms on a per patient and enterprise basis for screening any pathological condition per the clinical practice and to make transparent, understandable and explainable decisions.

With regard to the components and operations depicted in and described in accordance with the figures, any of the operations and sub-operations may be implemented as non-transitory computer-readable instructions stored on a computer-readable medium. The computer-readable instructions may, for example, be executed by the one or more processors of the sensor domain 20, patient app 21, and/or command center 22, as referenced herein, having a network element and/or any other device corresponding thereto, particularly as applicable to the applications and/or programs described above.

From the foregoing, it will be appreciated that various embodiments of the present disclosure have been described herein for purposes of illustration, and that various modifications may be made without departing from the scope and spirit of the present disclosure. Further, one of ordinary skill in the art would understand that the various embodiments may be combined or separated, and is within the possession of, consideration of, and contemplation of the inventors to combine various embodiments. Accordingly, the various embodiments disclosed herein are not intended to be limiting, with the true scope and spirit being indicated by the following claims.

One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

Furthermore, the present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and even apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

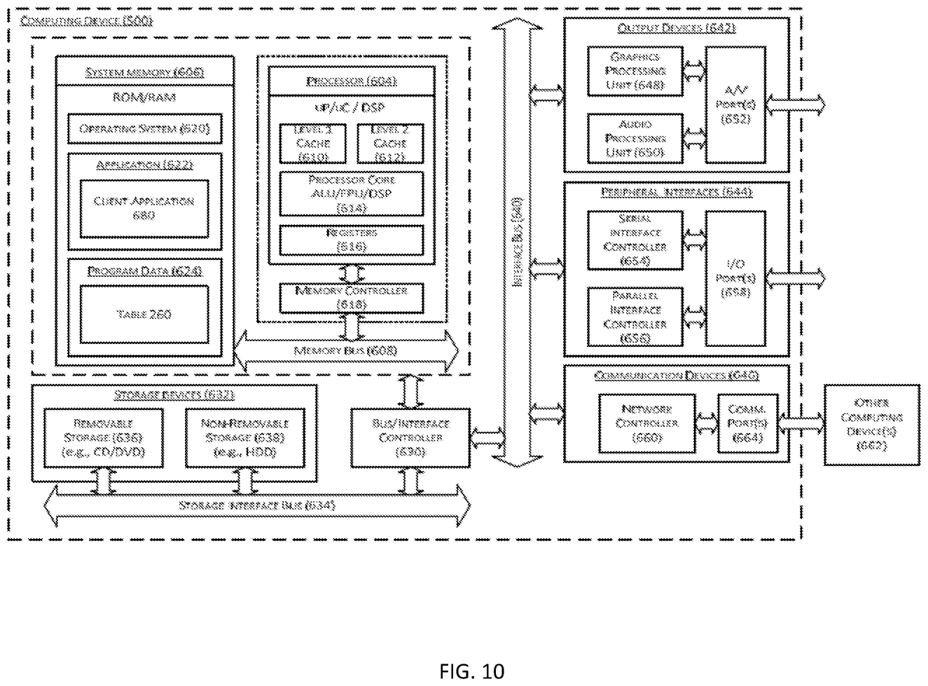

FIG. 10 shows sample computing device 600 in which various embodiments of the system and method for scalable clinician defined analytics may be implemented. More particularly, FIG. 10 shows an illustrative computing embodiment, in which any of the operations, processes, etc. described herein may be implemented as computer-readable instructions stored on a computer-readable medium. The computer-readable instructions may, for example, be executed by a processor of a mobile unit, a network element, and/or any other computing device.

In a very basic configuration 602, computing device 600 typically includes one or more processors 604 and a system memory 606. A memory bus 608 may be used for communicating between processor 604 and system memory 606.

Depending on the desired configuration, processor 604 may be of any type including but not limited to a microprocessor (μP), a microcontroller (μC), a digital signal processor (DSP), or any combination thereof. Processor 604 may include one more levels of caching, such as level one cache 610 and level two cache 612, processor core 614, and registers 616. An example processor core 614 may include an arithmetic logic unit (ALU), a floating point unit (FPU), a digital signal processing core (DSP Core), or any combination thereof. An example memory controller 618 may also be used with processor 604, or in some implementations memory controller 618 may be an internal part of processor 604.

Depending on the desired configuration, system memory 606 may be of any type including but not limited to volatile memory (such as RAM), non-volatile memory (such as ROM, flash memory, etc.) or any combination thereof. System memory 606 may include an operating system 620, one or more applications 622, and program data 624.

Application 622 may include Client Application 680 that is arranged to perform the functions as described herein including those described previously with respect to FIGS. 1-9. Program data 624 may include Table 650, which may alternatively be referred to as “figure table 650” or “distribution table 650,” which may be useful for measuring ECG, PCG, and ACC signals on a patient's skin surface as described herein.

Computing device 600 may have additional features or functionality, and additional interfaces to facilitate communications between basic configuration 602 and any required devices and interfaces. For example, bus/interface controller 630 may be used to facilitate communications between basic configuration 602 and one or more data storage devices 632 via storage interface bus 634. Data storage devices 632 may be removable storage devices 636, non-removable storage devices 638, or a combination thereof. Examples of removable storage and non-removable storage devices include magnetic disk devices such as flexible disk drives and hard-disk drives (HDD), optical disk drives such as compact disk (CD) drives or digital versatile disk (DVD) drives, solid state drives (SSD), and tape drives to name a few. Example computer storage media may include volatile and nonvolatile, removable and non-removable media implemented in any method or technology for storage of information, such as computer readable instructions, data structures, program modules, or other data.

System memory 606, removable storage devices 636, and non-removable storage devices 638 are examples of computer storage media. Computer storage media may include, but not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which may be used to store the desired information and which may be accessed by computing device 600. Any such computer storage media may be part of computing device 600.

Computing device 600 may also include interface bus 640 for facilitating communication from various interface devices, e.g., output devices 642, peripheral interfaces 644, and communication devices 646, to basic configuration 602 via bus/interface controller 630. Example output devices 642 may include graphics processing unit 648 and audio processing unit 650, which may be configured to communicate to various external devices such as a display or speakers via one or more A/V ports 652. Example peripheral interfaces 644 may include serial interface controller 654 or parallel interface controller 656, which may be configured to communicate with external devices such as input devices (e.g., keyboard, mouse, pen, voice input device, touch input device, etc.) or other peripheral devices (e.g., printer, scanner, etc.) via one or more I/O ports 458. An example communication device 646 may include network controller 660, which may be arranged to facilitate communications with one or more other computing devices 662 over a network communication link via one or more communication ports 664.

The network communication link may be one example of a communication media. Communication media may typically be embodied by computer readable instructions, data structures, program modules, or other data in a modulated data signal, such as a carrier wave or other transport mechanism, and may include any information delivery media. A “modulated data signal” may be a signal that has one or more of its characteristics set or changed in such a manner as to encode information in the signal. By way of example, and not limitation, communication media may include wired media such as a wired network or direct-wired connection, and wireless media such as acoustic, radio frequency (RF), microwave, infrared (IR) and other wireless media. The term computer readable media as used herein may include both storage media and communication media.

Computing device 600 may be implemented as a portion of a small-form factor portable (or mobile) electronic device such as a cell phone, a personal data assistant (PDA), a personal media player device, a wireless web-watch device, a personal headset device, an application specific device, or a hybrid device that include any of the above functions. Computing device 600 may also be implemented as a personal computer including both laptop computer and non-laptop computer configurations.

There is little distinction left between hardware and software implementations of aspects of systems; the use of hardware or software is generally (but not always, in that in certain contexts the choice between hardware and software can become significant) a design choice representing cost vs. efficiency tradeoffs. There are various vehicles by which processes and/or systems and/or other technologies described herein may be implemented, e.g., hardware, software, and/or firmware, and that the preferred vehicle may vary with the context in which the processes and/or systems and/or other technologies are deployed. For example, if an implementer determines that speed and accuracy are paramount, the implementer may opt for a mainly hardware and/or firmware vehicle; if flexibility is paramount, the implementer may opt for a mainly software implementation; or, yet again alternatively, the implementer may opt for some combination of hardware, software, and/or firmware.

The foregoing detailed description has set forth various embodiments of the devices and/or processes for determining patient body temperature via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In one embodiment, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a floppy disk, a hard disk drive, a CD, a DVD, a digital tape, a computer memory, etc.; and a transmission type medium such as a digital and/or an analog communication medium (e.g., a fiber optic cable, a waveguide, a wired communications link, a wireless communication link, etc.).

Those skilled in the art will recognize that it is common within the art to describe devices and/or processes in the fashion set forth herein, and thereafter use engineering practices to integrate such described devices and/or processes into data processing systems. That is, at least a portion of the devices and/or processes described herein can be integrated into a data processing system via a reasonable amount of experimentation. Those having skill in the art will recognize that a typical data processing system generally includes one or more of a system unit housing, a video display device, a memory such as volatile and non-volatile memory, processors such as microprocessors and digital signal processors, computational entities such as operating systems, drivers, graphical user interfaces, and applications programs, one or more interaction devices, such as a touch pad or screen, and/or control systems including feedback loops and control motors (e.g., feedback for sensing position and/or velocity; control motors for moving and/or adjusting components and/or quantities). A typical data processing system may be implemented utilizing any suitable commercially available components, such as those typically found in data computing/communication and/or network computing/communication systems.

The herein described subject matter sometimes illustrates different components contained within, or connected with, different other components. It is to be understood that such depicted architectures are merely examples, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include but are not limited to physically mateable and/or physically interacting components and/or wirelessly interactable and/or wirelessly interacting components and/or logically interacting and/or logically interactable components.

Lastly, with respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims, e.g., bodies of the appended claims, are generally intended as “open” terms, e.g., the term “including” should be interpreted as “including but not limited to,” the term “having” should be interpreted as “having at least,” the term “includes” should be interpreted as “includes but is not limited to,” etc. It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an,” e.g., “a” and/or “an” should be interpreted to mean “at least one” or “one or more;” the same holds true for the use of definite articles used to introduce claim recitations. In addition, even if a specific number of an introduced claim recitation is explicitly recited, those skilled in the art will recognize that such recitation should be interpreted to mean at least the recited number, e.g., the bare recitation of “two recitations,” without other modifiers, means at least two recitations, or two or more recitations. Furthermore, in those instances where a convention analogous to “at least one of A, B, and C, etc.” is used, in general such a construction is intended in the sense one having skill in the art would understand the convention, e.g., “a system having at least one of A, B, and C” would include but not be limited to systems that have A alone, B alone, C alone, A and B together, A and C together, B and C together, and/or A, B, and C together, etc. In those instances where a convention analogous to “at least one of A, B, or C, etc.” is used, in general such a construction is intended in the sense one having skill in the art would understand the convention, e.g., “a system having at least one of A, B, or C” would include but not be limited to systems that have A alone, B alone, C alone, A and B together, A and C together, B and C together, and/or A, B, and C together, etc. It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

What is claimed is:

1. A method for a scalable modular architecture for clinician defined analytics, comprising:
measuring, by one or more sensors of one or more devices, one or more clinical measurements of a patient;
aggregating, by a command center, the one or more clinical measurements;
extracting, by the command center, clinical measurement vectors from the aggregated clinical measurements for one or more decision system;
selecting, by the command center, appropriate clinical measurements relevant to triggering one or more decision systems from a pool of incoming data; and
performing computations, by the command center, to define an analytics engine per analytics specifications and operations.

2. The method of claim 1, wherein one or more decision support systems are triggered using the defined analytics engine.

3. The method of claim 2, wherein the defined analytics engine further performs evaluation tasks, optimization process, and computation process of the decision support system based on the definitions of the clinician.

4. The method of claim 3, wherein the evaluation tasks include evaluation and compilation of the definitions into a mathematical form used for computation, and verification of syntax involving automated compilation rules or a manual input table of operand values run through a mathematical form to ensure correct output.

5. The method of claim 4, wherein the optimization process includes minimization of states or declarative statements by reducing unreachable states or statements.

6. The method of claim 5, wherein the computation process uses data vectors for the operands and the optimized mathematical structure for computing the elements of the decision support system.

7. The method of claim 1, further comprising, defining and setting, by a clinician, the analytics specifications and operations.

8. The method of claim 7, wherein the defining and setting the analytics specifications and operation includes:
setting, by a clinician portal, processes of a decision layer for defining explicit explanatory mathematical structure of a decision support system;
defining operands required for the decision support system in the definition layer,
wherein the operand includes sensor inputs including at least one of vital sign measurements or lab results.

9. The method of claim 8, wherein the decision layer includes at least one of a Boolean process, scoring process, dynamic process, or mixed process.

10. The method of claim 9, wherein the Boolean process defines a decision structure whose result may trigger a single alert.

11. The method of claim 9, wherein the scoring process that defines a decision structure whose result generates scores that may trigger a multiple alert.

12. The method of claim 9, wherein the dynamic process defines a decision structure where the process transitions from one state to a next state until the process reaches a final state that may trigger a single alert.

13. The method of claim 9, wherein the mixed process is a dynamic process with each state being a Boolean process or a scoring process.

14. The method of claim 1, further comprising assisting a clinician in selecting definitions of the analytics specifications and operations by learning optimal decisions based on outcomes of prior definitions and operations.

15. The method of claim 1, further comprising integrating the one or more clinical measurements into an electronic health record (EHR).

16. A non-transitory computer-readable medium storing executable instructions for clinician defined analytics that, in response to execution, cause a computer to perform operations comprising:
measuring, by one or more sensors of one or more devices, one or more clinical measurements of a patient;
aggregating, by a command center, the one or more clinical measurements;
extracting, by the command center, clinical measurement vectors from the aggregated clinical measurements for one or more decision system;
selecting, by the command center, appropriate clinical measurements relevant to triggering one or more decision systems from a pool of incoming data; and
performing computations, by the command center, to define an analytics engine per analytics specifications and operations.

17. The non-transitory computer-readable medium of claim 16, further comprising:
defining and setting, by a clinician, the analytics specifications and operations, wherein the defining and setting the analytics specifications and operation includes:
setting, by a clinician portal, processes of a decision layer for defining explicit explanatory mathematical structure of a decision support system;
defining operands required for the decision support system in the definition layer,
wherein the operand includes sensor inputs including at least one of vital sign measurements or lab results.

18. The non-transitory computer-readable medium of claim 17, wherein the decision layer includes at least one of a Boolean process, scoring process, dynamic process, or mixed process.

19. The non-transitory computer-readable medium of claim 18,
wherein the Boolean process defines a decision structure whose result may trigger a single alert,
wherein the scoring process that defines a decision structure whose result generates scores that may trigger a multiple alert,
wherein the dynamic process defines a decision structure where the process transitions from one state to a next state until the process reaches a final state that may trigger a single alert, and
wherein the mixed process is a dynamic process with each state being a Boolean process

20. The non-transitory computer-readable medium of claim 16,
wherein one or more decision support systems are triggered using the defined analytics engine.
